(19) United States
(12) Patent Application Publication
(10) Pub. No.: US 2024/0110318 A1
(43) Pub. Date: Apr. 4, 2024

(54) REUSABLE MOISTURE-REGULATING TEXTILE SHEET

(71) Applicant: BODYVISION Technology GmbH, Mönchengladbach (DE)

(72) Inventors: Ellen BENDT, Mönchengladbach (DE); Christoph RICHTER, Mönchengladbach (DE); Towela CHISI, Mönchengladbach (DE); Ann-Kathrin BÜSCHKENS-GÖTZ, Mönchengladbach (DE); Lara ZAMPESE, Mönchengladbach (DE)

(73) Assignee: BODYVISION Technology GmbH, Mönchengladbach (DE)

(21) Appl. No.: 18/246,887

(22) PCT Filed: Oct. 4, 2021
(86) PCT No.: PCT/IB2021/022222

§ 371 Date: Mar. 28, 2023

(30) Foreign Application Priority Data
Oct. 1, 2020 (DE) 10 2020 006 022.6

(51) Int. Cl. D04B 1/12

(52) U.S. Cl. CPC D04B 1/12; D10B 2401/021; D10B 2401/022; D10B 2503/06; D10B 2509/026

(57) ABSTRACT

A textile sheet (1), which is in the form of a three-dimensional multi-layer knit structure having at least three layers, characterized in that the first and third layers each have at least two portions, wherein the first layer (2) absorbs the liquid and transports the liquid to the intermediate layer (3) wherein the first layer has an upper portion which is hydrophobic and a lower portion which is hydrophilic, both portions being smooth knitted and plated; the second layer (3), namely the intermediate layer (3), absorbs and retains the liquid, the intermediate layer having hydrophobic transverse threads (3a), which interconnect the first and third layers, and the intermediate layer having a hydrophilic inlay; and the third layer (4) prevents the liquid from escaping from the material wherein the third layer has an upper portion that is hydrophobic and a lower portion that is waterproof, both portion being smooth knitted and plated.

### CROSS-REFERENCE TO RELATED APPLICATIONS

This application is a National Stage application of International Patent Application No. PCT/IB2021/022222, filed Oct. 4, 2021, which claims priority to German Patent Application No. 10 2020 006 022.6, filed on Oct. 1, 2020, the disclosures of each of which is incorporated herein by reference in its entirety.

### TECHNICAL FIELD

The present invention relates to a textile sheet that has good liquid management.

### BACKGROUND OF THE INVENTION

Textile sheets, i.e. flat structures made of fibers or threads, are processed into knitted fabrics, knitwear, knitted goods, braids or also into wadding, non-woven fabrics and felt. The fibers or threads are knitted, felted, woven, crossed, glued and interlocked to be subsequently processed into fabrics, which are then further woven into cloths, for example dishcloths, or also further processed into clothing. High-quality functional textiles can also be produced, i.e. textiles made from fibers, threads, woven, knitted and crocheted fabrics with added functional value. These functional textiles can have the most diverse properties. They can be windproof, waterproof, breathable, thermoregulating, stain resistant, antimicrobial, flame retardant, UV resistant, electrically shielding, elastic, durable, easy to care for, chemical resistant, lightweight, warming/cooling, among others. The high number of properties listed here alone shows the great importance and demand for textiles, which should have special properties for specific purposes. Thus, functional textiles also combine qualities that one would not expect. The great popularity of breathable, i.e. permeable, and waterproof, i.e. impermeable to water, clothing for outdoor activities proves this.

The textile sheet taught here in this document is basically to have good liquid management. Liquid management means the receptacle of a quantity of liquid and the retention of this same quantity of liquid by the respective textile. Areas of application can be bed sheets, surgical drapes, incontinence sheets and hygiene and cleaning items. The body's loss of water during sleep, the draining of weeping areas of the body, the maintenance of mobility in cases of incontinence and the general need for hygiene and cleaning and their obvious consequences demand solutions.

Although there are already many products on the market that promise to remedy this situation, most of them still have room for improvement in many aspects.

In Germany, for example, an estimated six to eight million people suffer from urinary incontinence, which is also known colloquially as “weak bladder”. Approximately 200 million people worldwide are affected. Urinary incontinence is when urine passes uncontrollably and involuntarily. In this case, the body is missing or lacks the ability to store the contents of the bladder. Affected persons can no longer control when urine is passed. Urinary incontinence can affect even a young person. However, the number of people affected increases with age.

So-called absorbent (pads and diapers) or draining (e.g. catheters and condom urinals) incontinence products are offered as aids for those affected. Furthermore, there is still a large number of disposable products.

In this case, the development of a reusable incontinence sheet would be a solution to be pursued. The sheet to be developed must also have the different to contradictory properties already mentioned above. The main focus, of course, is on good liquid management. However, comfort of wear and use, washability, good olfactory properties and also sustainability, namely the absence of films and coatings that are difficult to dispose of, are also important properties to consider. The same requirements apply to bed sheets, which are designed to absorb body liquids, in this case the sweat of the users. For surgical drapes and dressings (e.g. wound dressings), additional requirements will have to be met, such as sterility. But here, too, liquid management is essential. In general, excretion of body liquids is a perfectly normal human property. However, in some situations, if you are not prepared, this can be inappropriate and therefore uncomfortable. Unwanted stains and odors from sweat, menstruation or incontinence can cause embarrassing feelings. Products available on the market consist of several layers, wherein the suction sheets of reusable products are often realized by sewing different materials together. These layers have to be connected to one another in a complex process. They can also lose the stored liquid again in the event of production errors, for example poor sewing.

### SUMMARY

It is therefore an object of the present invention to provide a solid textile sheet capable of absorbing, transporting and retaining liquid, and which is also reusable and washable. Furthermore, a method for manufacturing this textile sheet and its possible uses are given.

This object is solved by a textile sheet which is in the form of a three-dimensional multi-layer knit structure having at least three layers, characterized in that the first layer absorbs and transports the liquid to the intermediate layer, wherein said layer has a hydrophilic portion, the second layer namely the intermediate layer, receives and retains the liquid, wherein the intermediate layer has hydrophobic transverse threads connecting the first and third layers and is a hydrophilic inlay, and the third layer prevents the liquid from escaping the fabric, wherein the layer has a hydrophobic portion.

This textile sheet ensures that the various material properties are combined within the textile sheet. The first layer absorbs the liquid and transports it to the second layer, the intermediate layer. This first layer has a hydrophilic portion. This first layer is the one that, in the case of application to the human body, faces the body. It lies directly against the body. It ensures the rapid receptacle of the liquid. This hydrophilic portion is knitted smooth. It has the following properties, namely antibacterial, odorless and hypoallergenic. It can be made of any type of hydrophilic thread or hydrophilic fiber. The following fabrics are listed here, which are not to be understood as exhaustive: Tencel, Deocell, Deowool, cotton, modal, bamboo, lyocell, viscose and combinations thereof in any composition. The first layer thus has wicking and drying properties. In certain applications, the first layer may also have hydrophobic properties.

The second layer, also referred to as the intermediate layer because it lies between the first and third layers, has hydrophobic transverse threads. It itself serves as a hydrophilic inlay, which absorbs and retains the liquid. The hydrophobic transverse threads, however, connect the first and third layers in such a manner that they are attached to one another. The arrangement of the intermediate layer between the first and the third layer and the connection of the two layers via transverse threads can be made using catch handles. The intermediate layer itself is partly made of inlay fibers and/or inlay threads. They are also fixed by the transverse threads and thus held in their intended place. In fact, they are held there only by the transverse threads. Also the fabrics of the intermediate layer with hydrophilic character have antibacterial, odorless and hypoallergenic properties. They consist of any type of hydrophilic thread or hydrophilic fiber. The following fabrics with hydrophilic character are listed here, but are not to be understood as exhaustive: Tencel, Deocell, Deowool, cotton, modal, bamboo, lyocell, viscose, sodium polyarcylate, carbon crystals, monofilaments, multifilaments, loose fibers, microfibers, wood fibers, threads of superabsorbent fibers and combinations thereof in any composition. The transverse thread, i.e. the connecting thread that extends in the intermediate layer, is also antibacterial, odorless and hypoallergenic. It can be made of any type of thread with hydrophobic properties, such as polyester, synthetic monofilaments or multifilaments (PET, PES, PL, PCDT, PA, PP), but also natural fabrics such as linen or hemp.

The third layer, which prevents the liquid from escaping from the fabric, has a hydrophobic portion. It is the layer which faces away from the body in case of application to the human body. It does not come into contact with the body, unlike the first layer. It is also knitted smooth. Again, the material should have antibacterial, odorless and hypoallergenic properties. It can be made of any type of hydrophobic thread or hydrophilic fiber. The following fabrics are listed here, which are not to be understood as exhaustive: Wool, wool blends, cotton, polyamide, polyester, modal, tencel, optionally fusible thread and combinations thereof in any composition.

In this context, it is important to note that the inventive textile sheet can be divided into layers in terms of its operation. These layers are still divided into portions, but this textile sheet is single-layer. However, the present structure is knitted and can no longer be divided into its layers. It also does not require edging, so no edging is done during knitting or after the knitting process is complete. It is therefore a material that exists and can be used as a single-layer knitted fabric. This is not yet known in this form in the prior art. The prior art teaches, as mentioned above, functional sheets that are inserted into a fabric, by gluing, sewing, or otherwise. If the present inventive textile sheet is taken, it can be made up into any possible shape without having to be subsequently machined or even losing its function.

The inventive textile sheet is knitted, thus the final product is a knitted fabric or structure. It is therefore a looping of threads.

In a further embodiment of the textile sheet according to the invention, the textile sheet is characterized in that the first and third layers each have at least two portions, wherein the first layer has an upper portion that is hydrophobic and a lower portion that is hydrophilic, and the third layer has an upper portion that is hydrophobic and a lower portion that is waterproof. The two portions in the two layers (first layer and third layer) are each smooth knitted and plated. With the aid of the plating technique, the respective mesh layers are again extended by at least one further placed material layer. A second thread is brought into the respective knitting process.

The upper portion (plating thread) of the first layer, in turn, should have antibacterial, odorless and hypoallergenic properties. It can be made of any type of hydrophobic thread (fiber), such as synthetic materials like PET or PA or merino wool, wool blends or other hydrophobic materials and combinations thereof in any composition.

The lower portion (plating thread) of the third layer should also have antibacterial, odorless and hypoallergenic properties. The demand is thus omnipresent. It can be made of any type of hydrophobic thread (fiber), such as wool, wool blends optionally both felted or further optionally fusible thread, polyamide and combinations thereof in any composition. They may further be laminated and/or resin coated (leak proof) or otherwise coated (leak proof).

All the materials and material lists mentioned here are not to be understood as conclusive, they can be replaced by other functional materials. Any type of fiber or thread can occupy any layer or element of the structure described, depending on the area of application, the properties desired for the end product, and/or those properties that have proven to be economically advantageous to manufacturers. The structure of the present invention should be understood as a framework in which many variations can occur; parameters such as thickness, stitch density, material percentages, weight, etc. can vary as a function of the specific application. Similarly, materials can be appropriately combined to produce the desired properties for the appropriate applications, whether garments, incontinence, hygiene, human or veterinary, or otherwise. The present textile sheet according to the invention can be used alone or in combination with other fabrics, or it can also act as a membrane that is inserted into and fastened to other material constructs by sewing, gluing or other means.

The thickness or diameter of the textile sheet can be 2-3 mm. It is thus very thin. In particular uses, for example, the amount of filling material can increase the thickness of the textile sheet. Experiments have shown that a sheet with a thickness of 5 cm can be produced. The feel of the textile sheet is more like a textile fabric and not a diaper. This is desired when used as incontinence clothing.

Here once again descriptively the basic concept on which the subject matter of the inventive textile sheet is based. The object of the textile sheet with good liquid management, washability and associated reusability, as well as the odor-neutralizing properties, led to the multi-layer binding structures disclosed in the claims. Other challenges arose in this regard, such as comfort of wear and use, especially when the textile sheet is used in products that come into contact with the skin. The sheets on the body should be designed to be cuddly and soft, among other things. Thus, a textile sheet was required which would ensure rapid receptacle of liquid or a liquid medium and, on the other hand, would not allow the liquid or liquid medium to escape on the other side of the textile sheet. This can easily be shown by the example of a welding cloth. The sweat should be led away from the body, but not reach the clothing.

The inventive textile sheet is a washable and multi-layer knitted textile. It has highly absorbent properties. The combination of materials with opposite properties, namely hydrophobicity and hydrophilicity, results in porous layers throughout the composition that transfer aqueous fluids away from the body and towards the absorbent core of the knitted fabric. The layers and portions with their respective material and the physics of liquid transport has been detailed in the description and examples. The product compositions can be adapted to the respective fabric requirements. Additional textile finishing methods can be applied to improve the properties of the sheet. Thus, antibacterial and/or odor-neutral or odor-absorbing fibers can be knitted in. This can be done for each layer and portion individually or together. Thus, spinning in metal ion threads to increase the antibacterial character of the textile sheet is not a problem. High-quality carbon crystals and carbon meshes, as well as natural materials such as the odorless yam roots, can be part of the textile sheet and used in each layer (portion) of the knitted structure. This modular system allows problem-free expansion of the application and function options. Adaptation to different product groups is easily accomplished.

The textile sheet can be manufactured on a flat or circular knitting machine. The knitting method extends continuously. The three-dimensional multi-layer knit structure is created on the circular knitting machine or on the flat knitting machine by a determined sequence of stitches/loops (stitches) (i.e. stitches are created by the needle movement). A first layer (stitch) is knitted in the front needle bed on a flat knitting machine, and in the cylinder on a circular knitting machine. Both a hydrophobic and a hydrophilic thread are used here. The hydrophobic thread faces outward, so it faces the outer side of the textile sheet. The hydrophilic thread faces inwards, i.e. towards the inside of the textile sheet. The two threads are knitted simultaneously using a plating carrier (i.e., a thread guide) to produce a plated smooth stitch (stitch) on each needle, where the needles make one complete turn to produce a complete loop (stitch). After this, a second layer is knitted, which is knitted in the rear needle bed on a flat knitting machine and in the dial on a circular knitting machine. In this case, a hydrophobic thread or alternatively superimposed hydrophobic threads and low-melting thermoplastic thread (e.g. fusible thread) are used. In the case of the presence of the low-melting thermoplastic thread, it faces outward away from the needle bed or dial, while the hydrophobic thread faces inward. In the knitting process, a second thread can be included. This covers the identical path. The two threads are called base thread and plating thread. For the purposes of this application, the threads are given this meaning in context.

Either the single hydrophobic thread or superimposed hydrophobic threads and low melting thermoplastic thread (e.g. fusible thread) are knitted using a plating carrier (i.e. thread guide) to have a single stitch (a single stitch) on each needle where the needles make a complete revolution/movement to then create complete loops/loops (stitches). After this, the transverse thread(s) (pile thread(s)) is/are knitted into the textile sheet, wherein the transverse thread(s) (pile thread(s)) are knitted in the front and rear needle beds in the case of a flat knitting machine, and in the cylinder and dial in the case of a circular knitting machine. A standard carrier (standard pin or thread guide) is used for this purpose. The hydrophobic monofilament forming the transverse threads is placed between the needles, wherein only every 4th needle moves halfway in the front and rear needle beds (in the case of the flat knitting machine) or in the dial and cylinder (in the case of the circular knitting machine). This creates the handle that holds the first and third layers together. In order to create a zigzag binding image, the needles, which move, are each offset by 2 needles in the needle bed. Now the hydrophobic filling thread is placed between the layers. For this purpose, it is arranged (placed) between the front and rear needle beds (in the case of the flat knitting machine) or the cylinder and the dial (in the case of the circular knitting machine) by means of an insertion carrier (e.g. inlay or thread guide). The needles do not move during this process.

In other words, to further clarify the method, the knitting process on a knitting machine suitable for this purpose extends as follows: The needle from the front needle bed or the needle from the back needle bed goes up, when thread guiding with 2 yarns, both threads are placed in the needle. This process sequence is used for the 1st and 3rd layer. The transverse thread (pile thread, e.g. a monofilament) is inserted, every 4th needle moves half up and half down. The filling material is inserted.

This entire process is repeated until the textile sheet is finished knitting. The filling material cannot be removed from the textile sheet as it is held by the pile threads (transverse threads). Instead of every 4th needle guiding the transverse thread, every 3rd needle can be used. But here the transverse threads are closer together. If you take every 5th needle, you might have problems holding the filling material.

Of course, the inventive textile sheet will also be producible via a 3-D textile printer.

The inlay, i.e. the filling layer, and the monofilament, i.e. the hydrophobic transverse threads, actually form one layer, but it consists of 2 materials. However, in this application, the 2 materials are also named as 2 layers to clarify the effect. However, the meaning is clear from the context.

The use of such a textile sheet is manifold. It can be used wherever liquids or liquid media are to be absorbed and stored. Here, hygiene, odor neutrality, antibacterial properties, anti-allergic properties are other properties that are or can be inherent in the textile sheet. The structure of the textile sheet allows for modification, reinforcement and adaptation of individual layers to the respective application. The simple knitting process, which immediately creates the respective product, and the lack of post-processing (such as subsequent further sewing and application of edges) recommend its use in the medical field, in the care sector and in the field of hygiene and household hygiene. For example, they can be used in hospitals as surgical underlays, bed pads and dressing materials. The same applies to the veterinary medical sector. In particular, animal blankets, surgical underpads, dressing materials, menstrual pants should be mentioned here. It is also necessary to consider the use of the textile sheet in everyday life. Examples include curtains, tablecloths, covers for all types of seating furniture and bedspreads.

The textile sheet is manufactured by a method according to the invention, which is characterized in that in a first step S1 a first layer of a hydrophobic thread and a hydrophilic thread is knitted, wherein the hydrophobic thread faces outward and the hydrophilic thread faces inward, and in a second step S2 a second layer of a hydrophobic thread and/or a hydrophobic thread is knitted together with a low-melting thermoplastic thread wherein the hydrophobic thread faces inward and, in the case of the presence of the low-melting thermoplastic thread, faces outward, and subsequently in step S3 the transverse thread(s) is/are knitted, wherein only every 4th needle moves halfway through the knitting process. Then S4 the hydrophilic filling thread is placed between the two layers.

A further embodiment of the inventive method for manufacturing a reusable three-dimensional textile sheet (1) is characterized in that, after the first two steps, the transverse threads are knitted with offset. Thus, the attachment point of the pole threads is shifted against one another. The offset can assume any possible displacement of the pole thread layers with respect to one another. The sequence steps S1, S2, S3 and S4 are also not subject to a strict sequence plan, but can certainly run in different order. This also gives the textile sheet other qualities and properties. The pole threads, which in the following figures enclose an angle of 45° can also be knitted in a different angle measure. The different angular dimension is achieved by offsetting the attachment point of the handles between the textile sheets. The resulting fabric remains the same, of course, but uniformity and stability under pressure and other properties change.

Knitting and warp knitting are two different types of knitwear. Thus, they are textiles manufactured by knitting. Therefore, the manufacture of the textile sheet according to the invention can also be made as knitted fabric. The manufacturing method must be adapted. This means that for the purposes of this application knitting can also mean acting synonymously.

The entire method can be completed by a fulling process.

### Research Results

In Germany, an estimated six to eight million people suffer from urinary incontinence, also known colloquially as “weak bladder”. Approximately 200 million people worldwide are affected? Urinary incontinence is when urine passes uncontrollably and involuntarily. In this case, the body is missing or lacks the ability to store the contents of the bladder, and affected persons can no longer control themselves when urine is released. Even young people can be affected by urinary incontinence. However, the number of people affected increases with age.

There are a number of different causes that can lead to urinary incontinence. These can have neurological and/or organic causes. These include abdominal surgery, pregnancy, certain underlying diseases such as diabetes, and also nerve damage. Depending on the underlying causes, different forms of urinary incontinence are distinguished: Urge incontinence, stress incontinence, extraurethral incontinence, reflex incontinence, giggle incontinence, overflow incontinence and mixed incontinence.

In addition, different degrees of severity of incontinence are distinguished, depending on the amount of urine leaking unintentionally:
Severity of incontinence
Light incontinence: A few drops of urine between trips to the toilet up to 100 ml in 4 hours.
Recommended production suction capability: 150 ml to 300 ml.
Moderate incontinence: irregular output of urine up to 200 ml in 4 hours. Recommended production suction capability: 300 ml to 700 ml.
Severe incontinence: Very large bladder emptying of more than 200 ml in 4 hours.
Recommended production suction capacity: over 1000 ml.
Very severe incontinence: The entire contents of the bladder empties uncontrollably.
Recommended production suction capacity: over 1500 ml.

So-called absorbent (pads and diapers) or draining (e.g. catheters and condom urinals) incontinence products are offered as aids for those affected.

This present report focuses on the development of a reusable incontinence sheet. These sheets face the difficulty of developing a multi-layer sheet construction with different to opposing properties in the individual material layers. The main focus is on good liquid management (absorbing and retaining liquid), in combination with wear/use comfort, washability, good olfactory properties and, above all, a sustainable product (warping of films and coatings).

### 2. Testing of Disposable and Reusable Products

Testing of disposable and reusable products is performed following a standardized MDS test method. Test method no. 12/2015 MDS-Hi “Testing of absorbent incontinence aids”.

All test parameters were adjusted to the existing monitor sizes.

### 2.1 Test: Determination of Liquid Intake

When determining liquid intake, a urine substitute is taken as the liquid. This is intended to simulate the viscosity of urine in order to be able to test realistically. Liquid intake is an important indicator of product performance for incontinence products.

The test material is cut into 6 specimens a 4×8 cm and labeled.
a) The urine substitute is manufactured as a solution of 3.6 g salt and 400 ml water.
b) Determine dry sample weight.
c) Immerse specimen in urine substitute and weigh down with a 280 g pressure plate for 5 minutes.
d) Remove the sample from the urine substitute and place it on a sieve. Weigh down under the action of 3.2 kg on the pressure plate for one minute. Then weigh the sample.

### 2.2 Test: Determination of Liquid Output

A further test to determine product performance is the liquid output test. Incontinence aids should be used inconspicuously and comfortably in everyday life, which is why the test method must also be presented in a manner that is close to everyday life. Before this test can be performed, the previously used samples must be completely dry again, therefore there is a drying time of 48 hours (adjustment of room humidity and temperature).

### Method:

a) Mix 400 ml of water with 3.6 g of salt to manufacture urine substitute. Place the specimen in the test rack and pressurize with 15 ml urine substitute.
b) Weight the specimen with pressure pad and pressure plate (1.28 kg) for 5 minutes.
c) Weigh 4× filter paper with a sample size of 4×8 cm.
d) The experimental setup is stratified as follows: Sample->4× filter paper->pressure pad->weight.
e) The weight must venile on the test setup for 2 minutes. Then weigh the filter papers again.

### 3. Market Overview of Disposable Products

Light incontinence pads are usually made of disposable materials designed for single use only. The typical structure is as follows:
Starting on the body side:
Fleece cover
Gluing
Manifold support
Suction core; Cellulose plus superabsorbent granules
Gluing
Laundry protection film (mostly breathable)
Pressure sensitive adhesive
Silicone paper, peelable then lies on the side facing away from the body

The quality of an incontinence product typically depends, in addition to wearing comfort and discreet appearance, on the quality of the so-called absorbent core, which consists of a mixture of cellulose and so-called superabsorber. “It makes up about 70 percent of the total weight of an incontinence product and has varying levels of storage capacity. The cellulose fibers disperse the liquid, which the superabsorbent then absorbs. The superabsorbent consists of partially neutralized sodium polyacrylate (with skin-neutral pH). Its storage capacity for desalinated water is up to 500 times its own weight and for urine still 30 to 40 times. In the dry state, it is a granule. After receptacle of liquid, it is transformed into a gel by binding the liquid inside its structure. This provides a dry and skin-friendly feeling. Unpleasant odors are avoided and further reduced by the slightly acidic pH of the granules; this is mainly due to the inhibition of bacterial growth. A major component of the metabolism of the remaining bacteria is strong-smelling ammonia, which is also buffered by the low pH. This also applies to a limited extent to amines. Some products also contain additives to reduce odor. A further important quality factor for incontinence products, in addition to their low volume, is their high resistance to pressure. This is another advantage of polyacrylate over cellulose. Even under strong pressure, including when the body weight rests on the incontinence product while sitting or lying down, the material retains the liquid.

The first step was to test the properties of the disposable products commonly available on the market in order to obtain an overview of the properties of the various products. These are then later compared with the flour level products available on the market. Important in comparing the properties is the liquid intake and liquid output of the various disposable and reusable products.

### 5. New Development of Absorbent Sheet

### 3.1 Basic Concept for the Sheet Structure:

For the design of a sheet with the desired requirement profile, the reusable products commonly used on the market were examined and it was found that the suction sheets used are usually realized by sewing together different materials. This prefabricated layer structure is to be replaced with a multi-layer binding structure in combination with new materials. The challenge is to combine opposing material properties within one textile sheet: A rapid receptacle of liquid on the body side on the one hand and a leakage of liquid on the other binding side (towards the clothing) on the other hand. This is achieved by several different layers of material within a spacer fabric. The figure below shows schematically the different layers of the planned area in cross-section.

Basically, we are talking about 3 layers with different functions: For the outer layer (away from the body), a feltable wool is chosen, which is compacted (felted) in the subsequent finishing processes. This felting, in conjunction with the natural hydrophobic properties of wool, provides a waterproof sheet and is therefore suitable for the liquid-proof outer side. The side that is in contact with the body should transport the liquid as quickly as possible to the inside of the multilayer knitted fabric. In order to make the liquid-transporting body layer as thin as possible, the plating technique is used, by means of which two threads can again be positioned separately within the layer: The liquid-transporting thread is vented on the outside and the first liquid-retaining thread is vented on the inside. The liquid-transporting thread on the outside also serves to create the driest possible feeling on the skin and to prevent the return transport of liquid. The center layer is also designed from two threads: The so-called connecting thread, in this case a PES monofilament, has two functions in the multilayer knitted fabric to be developed here:
1. It connects the conductive body layer with the liquid-tight outer layer.
2. It is designed to provide dimensional stability of the sheet through metered stiffness to minimize unwanted liquid output (e.g., due to force applied while sitting).

It should be noted here: Excessive stiffness makes for an unacceptable feel, which is of high importance in incontinence products. The second material is the so-called weft material: The weft material is located between the body and outer layers as a spacer and absorbent. Preferably, low-twist rovings are used as weft material, which can have a high moisture absorption. The weft material is not subjected to mechanical stress as it is surrounded by body and outer layers. The direct body contact of the shot material is not given and skin sensory properties are therefore not relevant in this material layer. The low twist and the resulting strength of the weft material is necessary to be able to process the thread in the knitting process.

### 3.2 Overview of the Threads Tested in the Different Layers:

When selecting thread, the requirement profiles of an incontinence flour product as a whole and of the individual material layers described above must always be considered in detail. Hydrophilic and hydrophobic materials for moisture management are needed, as well as soft and firm materials for a balance between suppleness and stability. Further challenges lie in neutralizing odors as much as possible, since unpleasant odors should not develop when the product is worn. In addition to the naturally odor-neutralizing wool, the regenerated fiber Lyocell is of particular interest here, as its antibacterial properties have a favorable olfactory effect.

The possibility of manufacturing a thread from fibers with superabsorbent properties was also tested. The fiber properties make the spinning process very difficult, and the fibers could only be processed to a limited extent at all and only proportionally in blends with other materials; therefore, only initial trials were possible within the scope of this project.

### 5.1 Basic Concept for the Sheet Structure

The following thread distribution is the basis for the following development. It should be noted that there are several layers of material in the 3 functional layers:

Optionally, the outer layer can also be plated, resulting in a total of 6 layers.

The following threads were tested in the individual layers:

Version 1

| | |
|---|---|
| Body layer | External: Gramax, PES gray |
| | Interior: Genziana, wool black |
| Outer layer | Jawalan, wool gray |
| Weft material | Roving wool |
| Connecting thread | PES monofilament type 266 |

Version 2

| | |
|---|---|
| Body layer | External: Gramax, PES gray |
| | Interior: Genziana, wool black |
| Outer layer | Jawalan, wool gray |
| Weft material | Homemade thread |
| Connecting thread | PES monofilament type 266 |

Version 3

| | |
|---|---|
| Body layer | External: Gramax, PES gray |
| | Interior: Tencel (white) |
| Outer layer | Jawalan, wool gray |
| Weft material | Roving wool |
| Connecting thread | PES monofilament type 266 |

Version 4

| | |
|---|---|
| Body layer | External: Gramax, PES gray |
| | Interior: Tencel (white) |
| Outer layer | Jawalan, wool gray |
| Weft material | Homemade thread |
| Connecting thread | PES monofilament type 266 |

Version 6

| | |
|---|---|
| Body layer | External: Gramax, PES gray |
| | Interior: Tencel (white) |
| Outer layer | Jawalan, wool gray |
| Weft material | Tencel 100% |
| Connecting thread | PES monofilament type 266 |

Version 7

| | |
|---|---|
| Body layer | External: Gramax, PES gray |
| | Interior: Tencel (white) |
| Outer layer | Jawalan, wool gray |
| Weft material | Lyocell 70% Virgin wool 30% |
| Connecting thread | PES monofilament type 266 |

Version 8

| | |
|---|---|
| Body layer | External: Naturfit (53% PES + 47% Lyocell) |
| | Interior: Deowool (75% CO + 15% virgin wool + |
| | 10% Lyocell) |
| Outer layer | Jawalan, wool gray |
| Weft material | Lyocell 70% Virgin wool 30% |
| Connecting thread | PES monofilament type 266 |

Version 9

| | |
|---|---|
| Body layer | External: Naturfit (53% PES + 47% Lyocell) |
| | Interior: Deowool (75% CO + 15% virgin wool + |
| | 10% Lyocell) |
| Outer layer | Jawalan, wool gray |
| Weft material | Tencel 100% |
| Connecting thread | PES monofilament type 266 |

Version 10

| | |
|---|---|
| Body layer | External: Naturfit (53% PES + 47% Lyocell) |
| | Interior: Deowool (75% CO + 15% virgin wool + |
| | 10% Lyocell) |
| Outer layer | Jawalan, wool gray |
| Weft material | Lyocell 67% Cotton 33% |
| Connecting thread | PES monofilament type 266 |

Version 11

| | |
|---|---|
| Body layer | External: Naturtech (55% PES + 45% Lyocell) |
| | Interior: Deowool (75% CO + 15% virgin wool + |
| | 10% Lyocell) |
| Outer layer | Jawalan, wool gray |
| Weft material | Tencel 100% |
| Connecting thread | PES monofilament type 266 |

Version 12

| | |
|---|---|
| Body layer | External: Naturtech (55% PES + 45% Lyocell) |
| | Interior: Deowool (75% CO + 15% virgin wool + |
| | 10% Lyocell) |
| Outer layer | Jawalan, wool gray |
| Weft material | Lyocell 67% Cotton 33% |
| Connecting thread | PES monofilament type 266 |

Version 13

| | |
|---|---|
| Body layer | External: Naturtech (55% PES + 45% Lyocell) |
| | Interior: Deowool (75% CO + 15% virgin wool + |
| | 10% Lyocell) |
| Outer layer | Jawalan, wool gray |
| Weft material | Lyocell 70% Virgin wool 30% |
| Connecting thread | PES monofilament type 266 |

### 6.3 Developed Sheet

After some preliminary tests regarding feel and grip, 12 different versions were tested in the end. Here is the evaluation of 12 of the 13 material tests considered. Version 5 was discarded before testing began.

6.3.1 Liquid Intake

Versions 2 and 4, which can be seen in FIG. 61, were each produced with the self-produced superabsorbent thread and have a corresponding material thickness. Liquid intake is significantly increased by the use of superabsorbent fibers.

By using thinner weft threads in version 6-13, the feel of the textile sheet was significantly improved. The liquid transport inside, was improved by the use of PES thread.

### Liquid Intake/1 mm Specimen Thickness Developed Sheet (g)

### FIG. 62: Performance Factor Developed Sheet

When comparing the liquid intake per 1 mm specimen thickness, the two superabsorbent versions 2 and 4 no longer stand out enormously. Versions 1, 3, 6 and 7 show similar results and will be looked at in more detail for moisture release.

Comparison of liquid intake developed sheet and reusable products.

### Performance Factor Developed Sheet

### FIG. 63: Liquid Intake/1 mm Specimen Thickness Developed Sheet

FIG. 63 compares the performance value for the developed sheet and the reusable products. Sheets 1-4 are not considered further due to their stiffness/haptics. The manufacture of a novel superabsorbent thread requires more intensive investigation with regard to the blending ratio of superabsorbent fibers used in the yarn in order to achieve the best possible material properties. The two best results without superabsorbent thread are achieved by versions 6 and 7. Both versions have a polyester on the outside and a lyocell thread on the inside for the body layer. The only difference between the two versions is the weft material: Version 6 has 100% Tencel in the weft and version 7 has 70% Lyocell and 30% virgin wool.

### Liquid Output

### Liquid Intake/1 mm Specimen Thickness Developed Sheet (g)

### FIG. 62: Performance Factor Developed Sheet

When comparing the liquid intake per 1 mm specimen thickness, the two superabsorbent versions 2 and 4 no longer stand out enormously. Versions 1, 3, 6 and 7 show similar results and will be looked at in more detail for moisture release.

In terms of liquid output, it is again clear that the PES body layer brings a reduction in liquid output with versions 6 and 7. The comparison product Protechdry releases minimally more liquid. This output refers to the real liquid output and not to the performance value of the liquid output.

### Conclusion

We have succeeded in developing a material concept based purely on bonding technology that can be further developed for a variety of multi-level incontinence items and products with liquid management. The material concept shown should be understood as a modular system that can be adapted to different requirement profiles. The chosen variant in this present report can be implemented in different types of underwear. Knitted sheet technology also makes it possible to produce sheets with closed edges that are true to shape (fully fashioned or seamless). Additional manufacturing steps of the following products (cutting, sewing, trimming, sealing) can be saved, films and coatings become unnecessary. The spacer area of the developed sheet does not remain hollow, but is filled with a voluminous, moisture absorbing and binding weft thread.

### Forecast

The use of the textile sheet is intended for underwear and must be specified and adjusted again for each different form of underwear. For this, things like fit and cut still need to be worked out afterwards. Reusable incontinence pads must, of course, be washed several times. Therefore, the recommended sheets should be subjected to various washing tests. In addition, fluid uptake and delivery tests should be repeated after standardized wash cycles. Due to the use of naturally functional fibrous materials such as wool and lyocell, the product has good olfactory properties. Therefore, a comprehensive odor test should be performed to verify and classify the odor neutralization.

The research results have been adopted without mentioning any third party trademarks.

### BRIEF DESCRIPTION OF THE DRAWINGS

In the following, non-limiting embodiments o the present invention are discussed with reference to the drawing. In the drawings:

FIG. 1 shows schematically the basic structure of the textile sheet for use on the body;

FIG. 2a and FIG. 2b show schematically, basic structure of the textile sheet for use on the body with five layers of material and a detailed sketch of the first layer;

FIG. 3 shows schematically the basic structure of the textile sheet for use on the body with six layers of material;

FIG. 4 shows a schematic diagram of a method for manufacturing an embodiment of the textile sheet without plating; and

FIG. 5 shows the displacement of the pole threads in the individual sequence steps S3.

### DESCRIPTION OF THE PREFERRED EMBODIMENTS

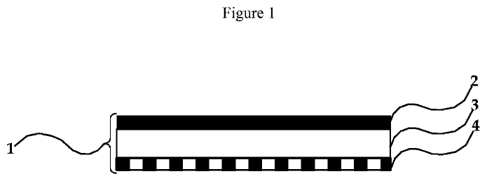

FIG. 1 shows schematically the basic structure of the textile sheet for use on the body.

Here it is easy to see the 3 functional layers from which a textile sheet 1 can be constructed. In this figure, the textile sheet 1 shows a 3-layer structure, wherein each of the layers performs a different function. For the outer layer 4, that is, the layer facing away from the body, in this case is chosen feltable wool. Felting provides densification of the material. Together with the hydrophobic, water-repellent properties of the wool, a waterproof sheet now exists. This makes the outer side 4 liquid-tight, so it is a blocking outer layer 4. The liquid present cannot leak to the outside of the clothing. The draining body layer 2, which lies directly against the body, is intended to transport the liquid produced as quickly as possible into the interior of the textile sheet 1. There is the absorbent and retentive layer 3, which absorbs and retains the liquid. The functions of these three layers are basically found in all embodiments. Thus, other technical features can introduce additional modifications and significant improvements. In order to make the liquid-transporting dissipative body layer 2 as thin as possible, the plating technique is used, by means of which two threads can again be positioned separately within the layer: The liquid-transporting thread is used on the outside and the liquid-retaining thread on the inside. The liquid-transporting thread also serves to create the driest possible feeling on the skin and prevent the return transport of liquid.

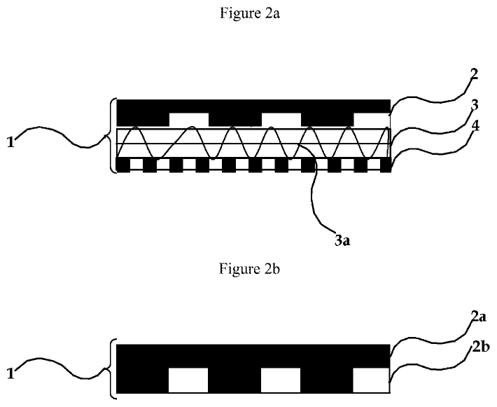

FIG. 2a and FIG. 2b schematically show the basic structure of the textile sheet according to the invention for use on the body with five material layers and a detailed sketch of the first layer. More features can now be seen in this schematic drawing. In principle, the number of functional layers mentioned above has remained the same. But the dissipative body layer 2, which is in direct contact with the body, is divided into two portions in this embodiment. It consists of an upper hydrophobic portion 2a and a lower hydrophilic portion 2b. The division into the two portions is indicated by the solid 2a or roughly gridded line 2b. In FIG. 2b, only the first layer 2 is shown enlarged. The upper portion 2a of the first layer is knitted smooth and plated with the lower portion 2b of the first layer. The lower portion 2b is also smooth knit. The upper portion 2a has hydrophobic properties, any kind of hydrophobic thread can be used for its structure. Synthetic materials such as PET or PA or merino wool, wool blends or other hydrophobic materials and combinations thereof in any composition have already been mentioned. PET, i.e. polyethylene terephthalate, as a textile fiber is crease-resistant, tear-resistant and weather-resistant. It hardly absorbs water due to its hydrophobic properties. PA, written out as polyamide, is also commonly known as nylon. It is characterized by its tensile strength and durability. Merino wool, which is also hydrophobic and a natural product, cools and absorbs odor particularly well. Accordingly, the hydrophilic portion 2b of the first layer is made of hydrophilic thread, such as Tencel, Deocell, Deowool, cotton, Modal, bamboo, Lyocell, viscose and combinations thereof in any composition. Tencel or Lyocell are derived from cellulose and have high dry and wet strength in fibrous form. They are soft and absorb moisture very well. These properties predestine this fabric to rest on the skin. The first layer 2 is, after all, the layer that is in contact with the body, that is, facing the body. As a processed Tencel fiber, Deocell is also antibacterial. Modal is also a cellulose product. However, it is smoother, more absorbent, more heat resistant and has higher strength compared to cotton or other fabrics. Rapid absorption of liquid on the side facing the body is possible due to the porous nature of the knitted fabric and the high absorption gradient formed by combining materials with opposite fiber properties in a single layer. The absorption process proceeds as follows: The upper portion of the first layer 2a is hydrophobic. It is directly facing the body, while the lower portion 2b is hydrophilic. This structure creates the high absorption gradient and the incoming liquid is quickly transported towards the second layer 3. Thus, the first layer 2 does not retain a significant amount of liquid and feels dry. Reflux towards the body, against the absorption gradient, is not possible. The absorbed liquid is no longer present near the body. This provides the high comfort of the present invention. It is also transported to a large extent by gravity into the second layer 3.

The second layer 3, also referred to as the intermediate layer because it lies between the first and third layers, has hydrophobic transverse threads 3a. Along these hydrophobic transverse threads 3a, the absorbed liquid is transported further, towards the absorbent core 3. This is highly hydrophilic. It consists of a hydrophilic filling thread and thus serves as a hydrophilic inlay that absorbs and retains the liquid. The filling thread is spun loosely and/or provided with additional microfibers. This provides an extended sheet with correspondingly high absorption capacity. Along the hydrophobic transverse threads 3a, the liquid also cannot return. The hydrophobic transverse threads 3a, however, connect the first layer 2 to the third layer 4 in such a manner that they are attached to one another. They are shown in this figure as a wavy line. The hydrophilic filling thread consists of Tencel, Deocel, Deowool, cotton, Modal, bamboo, Lyocell, viscose, sodium polyarcylate, carbon crystals, monofilaments, multifilament, loose fibers, microfibers, threads of superabsorbent fibers and combinations thereof in any composition. The hydrophobic transverse threads consist of synthetic monofilaments or multifilaments (PET, PA, PP) or natural materials such as linen or hemp.

There is a narrow gap between the first layer 2 and the second layer 3. This is created by the vertically extending transverse threads 3a. This space increases the breathability of the knitted fabric as a whole. In addition, faster absorption with subsequent drying of the absorbed liquid in the first layer 2 is supported. The liquid thus enters the absorbent core 3 even faster and remains there. The first and second layers 3 are thus also spatially separated by the narrow gap.

The third layer 4, the blocking outer layer, prevents the absorbed liquid from escaping from the knitted fabric. This layer is hydrophobic. It does not come into contact with the body. In this present embodiment, the layer consists of only one portion, namely wool, which is felted after the knitting process. However, other waterproof materials can be added to enhance the waterproof properties. The only important thing is to create a hydrophobic barrier.

In this embodiment, a washable, reusable, multi-faceted, knitted textile is present to ensure that unwanted liquids, moisture and odors are adsorbed away from the body, thereby promoting the health and hygiene needs of the wearer.

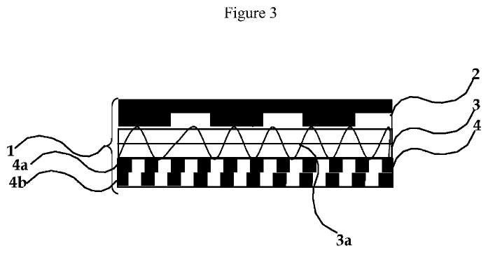

FIG. 3 schematically shows the basic structure of the textile sheet for use on the body with six material layers. In the upper part, FIGS. 2 and 3 are similar, but the third layer 4 may still be composed of 2 knitted layers (portions) 4a, 4b. This serves to increase moisture resistance when managing larger volumes of liquid. In addition to the portion (layer) 4a, which is made of hydrophobic material such as wool, another layer (portion) 4b is now added. In this embodiment, this further layer consists of a low-melting thermoplastic polymer. This thermoplastic polymer is knitted into the thread during the knitting process and then melted. This additionally closes and seals the pores of the knitted fabric once again. This measure therefore acts in addition to the felted wool, which indeed forms the inner portion 4a of the third layer (in this embodiment of the invention). This embodiment of the inventive textile sheet again clearly shows that a washable multi-layer knitted textile is present here. This has highly absorbent properties based on the combination of materials with opposing properties, constructed as a composition of porous layers that transfer aqueous fluids away from the body and towards the absorbent core of the knitted fabric. The layers and portions with their respective material and the physics of liquid transport has been in the description and examples. It should be noted here again that the product compositions can be adapted to the respective requirements. Textile finishing methods can be applied to further improve the sheet. Hygiene standards can be raised by inserting further antibacterial and/or odor-neutral or odor-absorbing fibers. This can be done for each layer and portion individually or together. Thus, spinning in metal ion threads to increase the antibacterial character of the textile sheet is not a problem. High-quality carbon crystals and carbon meshes, as well as natural materials such as the odorless yam roots, can be part of the textile sheet and used in each layer (portion) of the knitted structure.

The textile sheet can be knitted as one continuous piece on a flat bed or circular knitting machine. This means that the textile sheet can already be knitted in any desired shape, allowing possible finishing steps to be skipped or even omitted altogether. The knitting technique used here even allows complete made-up and shaped fabric parts to be incorporated into the final product. The method is environmentally friendly, economical and sustainable. In fact, there are no scraps, because here you do not have to cut. No further operations are necessary even after the knitting process. The product is completely finished. Among other things, it will no longer be possible to close fabric edges.

The following table gives the individual layers and portions, as well as their selection of the material to be used:

| sequence | sequence, detail | material | property |
|---|---|---|---|
| fluid-dissipating | plating thread 2a, | PET or PA or merino | hydrophobic |
| body layer 2, | upper portion | wool, wool blends or | |
| first layer | | other hydrophobic | |
| | | materials and | |
| | | combinations thereof | |
| | | in any composition | |
| fluid-dissipating | basic thread 2b, | hydrophilic thread, | hydrophilic |
| body layer 2 | lower portion | such as Tencel, | |
| first layer | | Deocell, Deowool, | |
| | | cotton, Modal, | |
| | | bamboo, Lyocell, | |
| | | viscose and | |
| | | combinations thereof | |
| | | in any composition. | |
| intermediate | filling thread 3a, | synthetic | hydrophobic |
| layer 3, | hydrophobic | monofilaments or | |
| second layer, | transverse thread | multifilaments (PET, | |
| center layer | | PA, PP) or natural | |
| | | materials such as linen | |
| | | or hemp | |
| intermediate | hydrophilic filling | Tencel, Deocel, | hydrophilic |
| layer 3, | thread, | Deowool, cotton, | |
| second layer, | hydrophilic Inlay | modal, bamboo, | |
| center layer | | lyocell, viscose, | |
| | | sodium polyarcylate, | |
| | | carbon crystals, | |
| | | monofilaments, | |
| | | multifilaments, loose | |
| | | fibers, microfibers, | |
| | | threads of | |
| | | superabsorbent fibers | |
| | | and combinations | |
| | | thereof in any | |
| | | composition | |
| third layer, | basic thread 4a, | wool, wool blends, | hydrophobic |
| blocking outer | | cotton, polyamide, | |
| layer 4 | | polyester, modal, | |
| | | tencel, optionally | |
| | | fusible thread (low | |
| | | melting thermoplastic | |
| | | thread) and | |
| | | combinations thereof | |
| | | in any composition | |
| third layer, | plating thread 4b | wool, wool blends | hydrophobic |
| blocking outer | | optionally both felted | |
| layer 4 | | or furthermore | |
| | | optionally felted | |
| | | thread, polyamide and | |
| | | combinations thereof | |
| | | in any composition | |

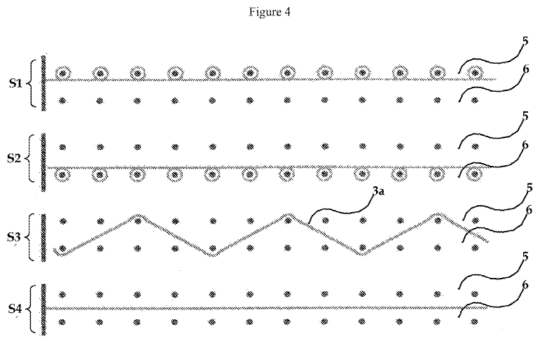

FIG. 4 shows schematically the method for manufacturing an embodiment of the textile sheet without plating. The textile sheet can be manufactured on a flat or circular knitting machine. In this embodiment, the textile sheet according to the invention is produced by a circular knitting machine. The three-dimensional multi-layer knit structure is created on the circular knitting machine by a determined sequence of stitches/loops (stitches). For this purpose, you can use an automatic knitting machine, which must be programmed.

The figure is constructed in such a manner that the chronological sequence is shown from top to bottom. You can clearly see the rear needle bed 5 and the front needle bed 6. The needles are represented as dots. In a first sequence step S1, a hydrophilic thread is knitted in the cylinder. For example, the thread guide runs from left to right. Hereafter, in a second sequence step S2, a hydrophobic thread is knitted in the dial. In the following third sequence step S3, the transverse threads 3a are knitted in. The hydrophobic monofilament is knitted into the cylinder and dial, wherein only every 4th needle is moved in this way. And thus created a handle. The hydrophilic filler material is then placed between the layers.

If the first and third layers are knitted in such a manner that the layers each consist of the 2 portions, then in a first sequence step S1, a hydrophilic thread and a hydrophobic thread are knitted in the cylinder, wherein the hydrophobic thread faces outward. The two threads are knitted simultaneously using a plating carrier (i.e., a thread guide) to form a plated smooth stitch on each needle, where the needles make one complete movement to create complete stitches. Hereafter, in a second sequence step S2, a hydrophobic thread and a low-melting thermoplastic (fusible thread) are knitted in the dial. The low-melting thermoplastic facing outward from the dial. In the following third sequence step S3, the transverse threads are knitted in. The hydrophobic monofilament is knitted into the cylinder and dial, wherein only every 4th needle is moved in such a manner that it does not pierce the sheets of the textile sheet, but only creates a handle. Subsequently, in sequence step S4, the hydrophilic filler material is placed between the layers. It cannot be removed from the textile sheet or fall out after the knitting process. The sequence steps S1 and S2 can also be performed in reverse order, followed by sequence step S3 and S4.

The transverse threads (pole threads) inserted in the 3rd sequencing step can also be offset from the already inserted pole threads in the subsequent sequencing step S3.

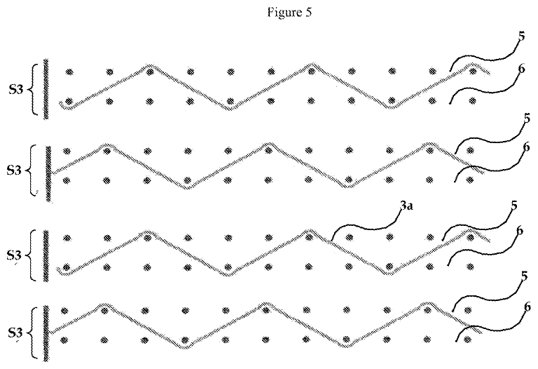

FIG. 5 shows the displacement of the pole threads in the individual sequence steps S3. In this case, you can see the displacement (offset) by one needle in needle bed 5. However, other displacements may also be possible. Such an offset provides a smoother sheet. This is particularly noticeable with firmer fabrics (textile sheets).

Although various embodiments of the present invention have been described and shown, the invention is not restricted thereto, but may also be embodied in other ways within the scope of the subject-matter defined in the following claims.

What is claimed is:

1. A textile sheet which is in the form of a three-dimensional multi-layer knit structure having at least three layers, wherein the first and third layers each have at least two portions, wherein
the first layer receives the liquid and transports it to the intermediate layer and wherein the first layer has an upper portion which is hydrophobic and a lower portion which is hydrophilic and both portions are smooth knitted and the portions are plated,
the second layer, namely the intermediate layer, absorbs and retains the liquid, wherein the intermediate layer has hydrophobic transverse threads connecting the first and third layers and is a hydrophilic inlay, and
the third layer prevents the liquid from escaping from the fabric and wherein the third layer has an upper portion that is hydrophobic and a lower portion that is waterproof and both portions are smooth knitted and the portions are plated.

2. (canceled)

3. The textile sheet in accordance with claim 1, wherein
the upper portion of the first layer is made of hydrophobic thread (fiber) selected from the following list, namely synthetic materials such as PET or PA or merino wool, wool blends or other hydrophobic materials and combinations thereof in any composition,
the lower portion of the first layer is made of hydrophilic thread (fiber) selected from the following list, namely Tencel, Deocell, Deowool, cotton, Modal, bamboo, Lyocell, viscose and combinations thereof in any composition,
the hydrophobic transverse threads are made of any type of hydrophobic thread, in particular polyester, synthetic monofilaments or multifilaments (PET, PA, PP), but also natural materials such as linen or hemp,
the hydrophilic inlay consists of any type of hydrophilic thread or fiber, in particular Tencel, Deocell, Deowool, cotton, Modal, bamboo, Lyocell, viscose, sodium polyarcylate, carbon crystals, monofilaments, multifilaments, loose fibers, microfibers, threads of superabsorbent fibers and combinations thereof in any composition,
the upper portion (ground thread) of the third layer is made of hydrophobic thread (fiber) selected from the following list, namely wool, wool blends, cotton, polyamide, polyester, modal, tencel, optionally fusible thread (low melting thermoplastic thread) and combinations thereof in any composition
the lower portion of the third layer is made of hydrophobic thread (fiber) selected from the following list, namely wool, wool blends optionally both felted or further optionally fusible thread, polyamide and combinations thereof in any composition.

4. A use of the textile sheet in accordance with claim 1 in the medical field, in particular as surgical underlays, bed pads and dressings, bandaging material.

5. The use of the textile sheet in accordance with claim 1 in the hygiene sector, in particular as incontinence or menstrual clothing, in particular incontinence pants and menstrual pants, underwear, sweat-absorbing textiles, rainwear.

6. The use of the textile sheet in accordance with claim 1 in the veterinary and animal hygiene sector, in particular animal blankets, surgical underlays, dressing material, menstrual pants.

7. The use of the textile sheet in accordance with claim 1 in the daily hygiene sector, in particular curtains, tablecloths, covers for seating furniture of all kinds, throw blankets.

8. A method for manufacturing a reusable three-dimensional textile sheet, wherein
in a first step S1, a first layer of a hydrophobic thread and a hydrophilic thread is knitted, wherein the hydrophobic thread faces outward and the hydrophilic thread faces inward, and
in a second step S2, a second layer of a hydrophobic thread and/or a hydrophobic thread is knitted together with a low-melting thermoplastic thread, wherein the hydrophobic thread faces inward and, in the case of the presence of the low-melting thermoplastic thread, faces outward,
following S3 the transverse thread(s) is/are knitted, wherein only every 4th needle moves halfway,
then S4, the hydrophilic filling thread is placed between the two layers.

9. The method for manufacturing a reusable three-dimensional textile sheet in accordance with claim 8, wherein after the first two steps the transverse threads are knitted with offset.

10. The method for manufacturing a reusable three-dimensional textile sheet, wherein the sequence steps S1, S2, S3, S4 follow one another in all conceivable sequences.

11. The use of the textile sheet in accordance with claim 3 in the medical field, in particular as surgical underlays, bed pads and dressings, bandaging material.

12. The use of the textile sheet in accordance with claim 3 in the hygiene sector, in particular as incontinence or menstrual clothing, in particular incontinence pants and menstrual pants, underwear, sweat-absorbing textiles, rainwear.

13. The use of the textile sheet in accordance with claim 3 in the veterinary and animal hygiene sector, in particular animal blankets, surgical underlays, dressing material, menstrual pants.

14. The use of the textile sheet in accordance with claim 3 in the daily hygiene sector, in particular curtains, tablecloths, covers for seating furniture of all kinds, throw blankets.
